(19) United States
(12) Patent Application Publication
(10) Pub. No.: US 2013/0079315 A1
(43) Pub. Date: Mar. 28, 2013

(54) Ophthalmic Gel Compositions

(71) Applicant: Martin J. Coffey, Pittsford, NY (US)

(72) Inventor: Martin J. Coffey, Pittsford, NY (US)

(21) Appl. No.: 13/600,723

(22) Filed: Aug. 31, 2012

Related U.S. Application Data
(63) Continuation of application No. 13/239,612, filed on Sep. 22, 2011.

(51) Int. Cl. A61K 47/30; A61P 27/02; A61K 31/56

(52) U.S. Cl. 514/178; 514/772.3

(57) ABSTRACT

A suspension comprising an ophthalmic active that has a solubility in water at 25° C. and a pH of 7 of less than 0.1 times the concentration of the active in mg/mL in the suspension, the ophthalmic active suspended in a formulation vehicle. The formulation vehicle comprises a lightly cross-linked carboxy-containing polymer and a concentration of ionic salt components to provide the suspension with a calculated ionic strength of less than 0.1. The suspension has the following rheological properties, G′>G″ and a suspension yield value of greater than 1 Pa. Also, upon addition of 30 mL of the suspension to a volume of 6 mL to 12 mL of simulated tear fluid, the resulting tear mixture transitions to a liquid form wherein, G″>G′ and the tear mixture has a yield value of less than 0.1 Pa.

### CROSS-REFERENCE

This application is a continuation application, and claims the benefit of U.S. patent application Ser. No. 13/239,612 filed on Sep. 22, 2011 under 35 U.S.C. §120, the entire contents of which are incorporated by reference herein.

### BACKGROUND

The present invention relates to a formulation vehicle for use with ophthalmic active agents that are insoluble in water to provide gel suspensions of the active agents during storage.

Ophthalmic compositions are used to provide relief of a variety of ocular conditions and ocular disease states. In most instances, ophthalmic compositions are administered or instilled to the eye via eye drops from a multi-dose container in the form of solutions, ointments or gels. If the ophthalmic active component is soluble, or even slightly soluble, in water, a formulator may proceed with a solution eye drop product. However, if the solution product has to low of a viscosity; e.g., less than about 30 cp (or mPa s), upon instillation the ophthalmic active can be rapidly discharged from the precorneal area of the eye because of lacrimal secretion and nasolacrimal drainage. As a result, it has been estimated that approximately 80-99% of the ophthalmic active component is simply washed or flushed from the eye before the active actually contacts the desired ocular tissue to achieve its desired clinical effect. The poor residence time of the active in the eye thus requires frequent instillation or use of a more concentrated active product to achieve the desired clinical effect. To lengthen the residence time of ophthalmic active, and thus, to enhance the bioavailability of the ophthalmic active per instillation, non-solution based ophthalmic vehicles have been developed. Examples of such ophthalmic vehicles include ointments, suspensions, and aqueous gels. However, these ophthalmic vehicles can have their drawbacks as well. For example, the use of ointments often causes blurred vision just after instillation. In some instance, the patient can sense a “goopy feeling” in their eyes, which, of course, is also undesirable.

Some ophthalmic formulators have resorted to the so-called in situ gel-forming systems. These ophthalmic vehicles can extend precorneal residence time and improve ocular bioavailability of the ophthalmic active. Typically, in situ gel-forming systems are usually aqueous solutions and contain one or more polymers. The ophthalmic products tend to exist as a low-viscosity liquid during storage in the dispenser container and form a gel upon contact with tear fluid. The liquid-to-gel transition can be triggered by a change in temperature, pH, ionic strength, or the presence of tear proteins depending on the particular polymer system employed.

For example, A. Rozier et al., Int. J. Pharm. (1989), 57: 163-168, discloses a composition comprising an ion-activated gelling gellan gum (a polysaccharide) with the tradename of Gelrite® and an ion content below the gelation concentration. Rozier et al.'s gellan gum composition rapidly gels when mixed with simulated tear fluid having a combined concentration of mono- and divalent cations (sodium and calcium) of about 0.14 M. U.S. Pat. No. 5,192,535 discloses an aqueous ophthalmic composition comprising a crosslinked carboxy-containing polymer. The composition has viscosity in the range of 1,000-30,000 cp and pH of 3-6.5, which rapidly gels (to viscosity of 75,000-500,000 cp) upon contact with the higher pH of tear fluid. Joshi et al.'s U.S. Pat. No. 5,252,318 discloses reversibly gelling aqueous compositions which contain at least one pH-sensitive reversibly gelling polymer (such as carboxy vinyl linear or branched or cross-linked polymers of the monomers) and at least one temperature-sensitive reversibly gelling polymer (such as alkylcellulose, hydroxyalkyl cellulose, block copolymers of polyoxyethylene and polyoxypropylene, and tetrafunctional block polymers of polyoxyethylene and polyoxypropylene and ethylenediamine). It is contemplated that a high amount of salt (up to 0.2-0.9%) is used to have a low viscosity in the ungelled state. The compositions are formulated to have a pH of 2.5-6.5; preferably, 4-5.5. The viscosity of the compositions increases by several orders of magnitude (up to 1,000,000 cp) in response to substantially simultaneous changes in both temperature and pH.

U.S. Pat. No. 6,511,660 discloses a composition comprising Carbopol and Pluronic® (a polyoxyethylene-polyoxypropylene copolymer) formulated at pH of 4. The composition turns into a stiff gel when in contact with physiological condition (37° C. and pH of 7.4). Kumar et al., J. Ocular Pharmacol., Vol. 10, 47-56 (1994), discloses an ocular drug delivery system based on a combination of Carbopol and methylcellulose, prepared at pH of 4. This system turns into a stiff gel when the pH is increased to 7.4. Kumar et al., J. Pharm. Sci. Vol. 84, 344-348 (1995), discloses yet another ocular drug delivery system containing Carbopol® and hydroxyproplymethylcellulose, also prepared at pH of 4. This system turns into a stiff gel when the pH is increased to 7.4 and the temperature to 37° C. In both systems, a viscosity-enhancing polymer (methylcellulose or hydroxypropylmethylcellulose) is added in order to not have excessive amount of Carbopol® concentration without compromising the in situ gelling properties as well as overall rheological behaviors. Finkenaur et al.'s U.S. Pat. No. 5,427,778 discloses gel formulations that contain a polypeptide growth factor and a water soluble, pharmaceutically or ophthalmically compatible polymeric material for providing viscosity within various ranges determined by the application of the gel.

The above prior-art ophthalmic compositions all have a common characteristic of having a low viscosity in the dispenser container and becoming a stiff gel upon being instilled in the eye due to an increase in at least one of pH, temperature, and ionic strength. Although a stiff gel can have an extended residence in the eye and assist in promoting a higher drug bioavailability, and perhaps enhance clinical outcome per instillation, such gels, like the ointments, can interfere adversely with vision and result in patient dissatisfaction. In addition, these prior-art compositions must often be formulated at significantly acidic pH, which is not comfortable upon installation in the eye of the patient.

In some instances, the ophthalmic active is virtually, or completely, insoluble in an aqueous solution-based formulation. For example, U.S. Pat. Nos. 5,538,721 and 4,540,930 describe a pharmaceutical composition comprising an amino-substituted steroid therapeutic agent, and an effective stabilizing amount of lightly cross-linked carboxy-containing polymer. Cyclodextrin is also used to least partially solubilize the therapeutic agent, in an aqueous medium.

### SUMMARY OF THE INVENTION

A suspension comprising an ophthalmic active that has a solubility in water at 25° C. and a pH of 7 of less than 0.1 times the concentration of the active in mg/mL in the suspension, the ophthalmic active suspended in a formulation vehicle, the vehicle comprising a lightly cross-linked carboxy-containing polymer and a concentration of ionic salt components to provide the suspension with a calculated ionic strength of less than 0.1. The suspension has the following rheological properties, G′>G″ and a suspension yield value of greater than 1 Pa. Also, upon addition of 30 mL of the suspension to a volume of 6 mL to 12 mL of simulated tear fluid, the resulting tear mixture transitions to a liquid form wherein, G″>G′ and the tear mixture has a yield value of less than 0.1 Pa.

A suspension comprising an ophthalmic active that has a solubility in water at 25° C. and a pH of 7 of less than 0.1 times the concentration of the active in mg/mL in the suspension, and the ophthalmic active is suspended in a formulation vehicle, the vehicle comprising a lightly crosslinked carboxy-containing polymer and a concentration of ionic salt components to provide the suspension with a calculated ionic strength of from 0.03 to 0.08. The suspension has the following rheological properties, G′>G″ and a suspension yield value of from 2 Pa to 8 Pa. Also, upon addition of 30 mL of the suspension to a volume of 10 mL of simulated tear fluid to provide a tear mixture of the suspension in a simulated ocular condition, the tear mixture has a tear mixture yield value from 0 Pa to 0.1 Pa and a tear thin value of from 5 to 30.

A method for suspending an ophthalmic active that has a solubility in water at 25° C. and a pH of 7 of less than 0.1 times the concentration of the active in mg/mL in an aqueous-based, ophthalmic suspension. The method comprises combining the ophthalmic active with a formulation vehicle, the vehicle comprising a lightly crosslinked carboxy-containing polymer and a concentration of ionic salt components to provide the suspension with a calculated ionic strength of from 0.03 to 0.08. The suspension has the following rheological properties, G′>G″, a suspension yield value of from 2 Pa to 8 Pa, and upon addition of 30 mL of the suspension to a volume of 10 mL of simulated tear fluid to provide a tear mixture of the suspension in a simulated ocular condition, the tear mixture has a tear mixture yield value of less than 0.1 Pa and a tear thin value of from 5 to 30.

A unit dosage package for administration of an ophthalmic formulation in the form of an eye drop, the ophthalmic formulation comprising an ophthalmic active that has a solubility in water at 25° C. and a pH of 7 of less than 0.1 times the concentration of the active in mg/mL in the formulation. The ophthalmic active is suspended in a formulation vehicle, the formulation vehicle comprising a lightly crosslinked carboxy-containing polymer and a concentration of ionic salt components to provide the suspension with a calculated ionic strength of from 0.03 to 0.08. The ophthalmic formulation has the following rheological properties, G′>G″, and a suspension yield value of from 2 Pa to 8 Pa, and upon addition of 30 mL of the suspension to a volume of 10 mL of simulated tear fluid to provide a tear mixture of the suspension in a simulated ocular condition, the tear mixture has a tear mixture yield value from 0 Pa to 0.1 Pa and a tear thin value of from 5 to 30.

An improved ophthalmic formulation over the current Alrex® formulation. The ophthalmic formulation contains less active, 0.16 wt. % loteprednol etabonate vs. the 0.2 wt. % loteprednol etabonate in the Alrex® product. More importantly, a small clinical study indicates that the ophthalmic formulation (taken once daily) is just as or more effective in reducing ocular itching for the treatment of seasonal allergic conjunctivitis than Alrex® (taken 4× per day). In other words, a once daily, drop administration of the ophthalmic formulation (0.16 wt. %) is more effective than 4×0.2 wt. % for a total administration of 0.8 wt. % of Alrex®.

### BRIEF DESCRIPTION OF THE DRAWINGS

FIG. 1 is the shear plots of CE2 (Besivance™) formulated with Durasite® vehicle at different dilutions with simulated tear fluid.

FIG. 2 is the shear plots of the LE formulation of Example 2 at different dilutions with simulated tear fluid.

### DETAILED DESCRIPTION OF THE INVENTION

Due to the unique physiological and biomechanical conditions of the eye formulating ophthalmic compositions to optimize clinical efficacy and patient compliance, yet minimize or avoid patient dissatisfaction following instillation in the form of drops remains a great challenge. The challenge is heightened considerably with ophthalmic actives that are insoluble in water. Due to the limited, or near non-existent, solubility of the active in water, the active must be suspended in a vehicle, typically as an emulsion or ointment. In such instances, however, it is very difficult to formulate ophthalmic actives to maintain a substantially uniform suspension or distribution in the formulation vehicle in order to have a consistent unit (instillation) dosage. In nearly all instances, a patient will have to shake the product (much like an inhaler used by asthma patients) to best ensure a consistent and accurate dosage. For this reason, it is especially difficult to suspend a water insoluble ophthalmic active in ophthalmic formulation for drop instillation that does not require a pre-shaking of the product. The formulation vehicle described herein addresses these shortcomings with present ophthalmic suspension formulations.

As used herein, use of the term the “solubility in water” of an ophthalmic agent in water, means the agent has a solubility in water as measured at 25° C. and pH of 7 of less than 0.1 times the concentration of the active in mg/mL in the ophthalmic formulation. For example, if the ophthalmic active is present in an ophthalmic formulation at a concentration of 0.1 mg/mL, the ophthalmic active will have a solubility in water at 25° C. and a pH of 7 of less than 0.1 (0.1 mg/mL), which is less than 0.01 mg/mL. Likewise, for an ophthalmic active that is present in an ophthalmic formulation at a concentration of 10 mg/mL, the ophthalmic active will have a solubility in water at 25° C. and a pH of 7 of less than 0.1 (10 mg/mL), which is less than 1.0 mg/mL. Accordingly, the term “solubility in water” refers to the water solubility of a specific agent in the suspension as well as the agent's concentration in the suspension in mg/mL. In other words, an ophthalmic active present at a relatively high concentration in a suspension can have a somewhat greater water solubility than another agent with a lower water solubility present in another suspension at a lower concentration, but because of the higher concentration in the former suspension a significant portion of the former agent remains suspended in the formulation.

The described ophthalmic formulation vehicle provides a storage-stable, suspension of an ophthalmic active in the form of a gel. However once instilled into the eye via one or more eye drops, the gel transitions to a liquid form, i.e., it loses its gel character. This transition from gel to liquid is important for patient compliance because of the dissatisfaction patients express after having instilled ophthalmic gels or ointments. These prior art vehicle formulations remain for a period of time in the eye as gels, particularly over the initial 1 to 3 minutes following instillation, and cause visual impairment. The gels or ointments can also cause ocular discomfort, which can lead to patients skipping one or more of a scheduled dosing regimen. The term “storage-stable” means that a stirred or shaken preparation of an ophthalmic active and the described formulation vehicle will provide a suspension of the active in the formulation vehicle, and the active will remain effectively suspended in the formulation vehicle for at least two weeks, and in many cases, for up to four weeks or even eight weeks, without having to stir or shake the drug product in its packaged container. The term “effectively suspended” means a drug suspension formulation that delivers 90% to 110% of a predetermined dosage of pharmaceutical active per eye drop without a patient having to purposefully shake the drug product container more than once every two weeks. Why is the storage-stability of an ophthalmic suspension so important? Because with non-storage-stable formulations many patients forget to shake the product before instillation. As a result, the patient is not instilling a consistent and proper dosage. This can be a problem because after the first twenty drops or so each subsequent eye drop can contain greater concentrations of ophthalmic active, which may not be a good thing.

In addition, to the described formulation vehicle having the characteristics of transitioning to a liquid upon instillation to the eye, in the case of a suspension of the ophthalmic active, Loteprednol Etabonate (at times referred to as LE), the described vehicle formulation provides the same or better clinical outcomes as presently marketed LE suspensions and LE ointments. In other words, one can actually formulate with the same, or even a smaller, concentration of LE and still achieve identical clinical outcomes. This comes as quite a surprise because it is presently believed by persons of skill in the art that formulations that exist as gels in the eye (i.e., they retain the viscoelastic property of a gel) would have greater residence time in the ocular environment, and therefore, provide greater bioavailability. The belief is that it is takes considerably more time for tear fluids to cause wash out of the ophthalmic formulation in the case of a gel compared to a formulation that exists in a liquid state in the eye. That long held belief is now shaken.

The described formulation vehicle is used to provide a suspension of an ophthalmic active that has a solubility in water at 25° C. and a pH of 7 of less than 0.1 times the concentration of the active in mg/mL in the formulation. The vehicle formulation comprises a lightly cross-linked carboxy-containing polymer and a concentration of ionic salt components to provide the suspension with a calculated ionic strength of less than 0.1. In addition, the suspension has the following rheological properties, G′>G″ and a suspension yield value of greater than 1 Pa. e.g., from 2 Pa to 10 Pa or from 3 Pa to 6 Pa. Also, upon addition of 30 mL of the suspension to a volume of 6 mL to 12 mL of simulated tear fluid, the resulting tear mixture transitions to a more liquid-state wherein, G″>G′ and the tear mixture has a yield value of less than 0.1 Pa, and more likely less than 0.05 Pa or less than 0.01 Pa. In fact, in many instances, the mixture will have no measurable yield value using the experimental methods described in Example 1 under the subheading, Experiments Rheology.

Viscoelasticity of a compositional fluid can in-part be described by what is referred to as a complex shear modulus defined by the following formula.
G*=G′+iG″ where i²=−1, and G′ is referred to as a storage modulus, and G″ is referred to as a loss modulus.
   G′ is often referred to as the elastic modulus or storage modulus and relates to the fluid's ability to store elastic energy. G″ is often referred to as the viscous modulus or loss modulus and relates to the fluid's viscosity or its ability to dissipate energy when a shear force is applied to the fluid. When G″>>G″, then the viscoelastic properties are dominated by the elastic properties and indicates that the composition is classified as a gel (semisolid). When G″>G′, then the viscoelastic properties are dominated by the viscous behavior and the composition is classified as a sol (liquid).

The relative magnitude of G′ and G″ can also be quantitated using an angle delta, δ, which is defined by tan δ=G″/G′. Under conditions where G′=G″, then tan δ=1 and δ=45 degrees. When G′>>G″ then tan δ<<1 and δ<45 degrees. For vehicle formulations that exhibit the desired behavior of maintaining a physically stable suspension in the bottle, δ is less than 10° and tan δ less than 0.2. Many of the preferred vehicle formulations will exhibit a δ is less than 6°, e.g., from 2° to 5°, or a tan θ of less than 0.105, e.g., a tan δ from 0.035 to 0.0875, respectively. This requirement that tan δ be less than 0.2 ensures that G′ is at least 5 times greater than G″. Because the values of G′ and G″ may be affected by the oscillatory frequency used in the measurement, it is preferred that the values of δ and tan δ be measured at 1 rad/s.

The simulated tear fluid used to determine the rheological properties of the mixture is listed in Table 1 below. Hanks balanced salt solution (Gibco HBSS with calcium and magnesium. P/N 14025, Invitrogen Corp, Carlsbad, Calif.) was used as simulated tear fluid. Again, addition of the tear fluid is used to simulate the ocular environment of the suspension following instillation into the eye. If you calculate the ionic strength of the simulated tear fluid one obtains a value of 0.15. The ionic strength is essentially equivalent to the ionic strength of 0.9% saline, which is also calculated to be 0.15. This is expected because the simulated tear fluid is comprised of 0.8% saline, and the additional salts and buffer agents make a small contribution to the ionic strength to the solution.

TABLE 1

| Component | mg/L | mmol |
|---|---|---|
| Calcium chloride | 140 | 1.26 |
| Magnesium chloride | 100 | 0.493 |
| Magnesium sulfate | 100 | 0.407 |
| Potassium chloride | 400 | 5.33 |
| Potassium dihydrogen phosphate | 60 | 0.441 |
| Sodium hydrogen carbonate | 350 | 4.17 |
| Sodium chloride | 8000 | 138 |
| Disodium hydrogen phosphate | 48 | 0.338 |
| d-glucose | 1000 | 5.56 |

In many embodiments, the suspension will have a viscosity in the container for instillation of eye drops of from 1000 cp to 2000 cp, and the calculated ionic strength is from 0.03 units to 0.1 units. Viscosity is measured with a Brookfield Engineering Laboratories LVDV-III Ultra C rheometer (a cone-and-plate rheometer) with CPE-52 spindle, at 25° C., and shear rate of 7±1 s⁻¹. Additional information on the viscosity measurements is described in the Example section.

Controlling the ionic strength of the suspension with the formulation vehicle is important to achieve the desired rheological properties. Accordingly, the suspension will have a calculated ionic strength from 0.03 units to 0.1 units, preferably from 0.05 units to 0.09 units.

The ionic strength of the formulation is calculated according to the standard equation that states:

$$μ = \frac{1}{2} {\sum }_{i} {C}_{i} {z}_{i}^{2} .$$

where the ionic strength, μ, is ½ the sum, over all charged species, i, of the product of the molar concentration, Cᵢ, and the square of the ion charge, zᵢ. The ionic strength is calculated using the equilibrium concentration of charged species at the pH of the formulation and not based solely on the formulation recipe. The equilibrium concentration of charged species for formulation components, at the pH of the formulation, can be estimated using the dissociation exponents, pK, for the ionizable species and the Henderson-Hasselbach equation. More preferably, the equilibrium concentration of charged species and ionic strength will be calculated simultaneously using an iterative approach similar to that outlined in Okamoto et al. [H. Okamoto, K. Mori, K. Ohtsuka, H. Ohuchi, and H. Ishii. Pharmaceutical Research, Vol. 14, No. 3, 1997] where a computer program is used to also include the effect of ionic strength on the pK. The contribution of the cross-linked carboxy-containing polymer to the ionic strength is included by treating the polymer as simply composed of acrylic acid with a monomer weight of 72 g/mol and a pKa of about 4.5. For example, at pH below the pKa, the polymer does not significantly contribute to the ionic strength, but at pH above the pKa, sodium acrylate (present when the polymer is neutralized by addition of sodium hydroxide) will contribute to the ionic strength.

The relatively fast transition from gel to liquid upon instillation of a described suspension into an eye is an important rheological characteristic of the ophthalmic formulation vehicle. We believe that this transition from gel to liquid may be triggered by the sudden change in ionic strength resulting from the dilution of the suspension with small amounts of tear fluid, particularly within the initial five minutes following instillation. The ionic strength of tear fluid is relatively quite high because of the high salt concentrations in tears. As the ophthalmic suspension mixes with the tear fluid the ionic strength of the resulting suspension-tear mixture, hereafter tear mixture, increases relative to the suspension and causes the suspension to thin. Also, because the concentration of the carboxy-containing polymer in the described vehicle formulation is typically less than the formulation vehicle of prior art drug suspensions the increase in the ionic strength upon dilution with tear fluid has greater affect and leads to greater tear thinning. The relatively large difference in the pre- and post-instillation ionic strength environments, and the relatively smaller amounts of carboxy-containing polymer in the vehicle formulations described herein, is believed to drive the thinning of the suspension in the eye.

As stated, the tear thinning characteristics of the described vehicle formulations is driven primarily by the differences in the ionic strength between the vehicle formulation, which has a low ionic strength relative to the high ionic strength of tear fluid, represented by the simulated tear fluid. As the vehicle formulation is administered in the form of an eye drop onto an eye, the percentage increase in the ionic strength of the tear mixture causes the vehicle formulation to thin. Accordingly, one can define a % ionic strength as a percent ratio of the ionic strength of the vehicle over the ionic strength of the simulated tear fluid, which is 0.154. Because the concentration of polymer also plays a role in the thinning characteristics of the vehicle formulations one can multiply the polymer concentration by the % ionic strength to give a tear thin value that can be used to predict with some confidence whether one will observe the desired tear thinning of a vehicle formulation.

% ionic strength=[formulation i.s./tear i.s.]×100

tear thin value=% ionic strength×[polymer, wt. %]

Accordingly, in one embodiment, the ophthalmic suspension will comprise an ophthalmic active that has a solubility in water at 25° C. and pH of 7 of less than 10% of the formulated concentration, which is suspended in a formulation vehicle. The vehicle will have from 0.3 wt. % to 0.5 wt. % of a cross-linked carboxy-containing polymer described below, and from 0.01 wt. % to 0.2 wt. % of sodium and/or potassium salts. The suspension can also include small amounts of bivalent calcium/magnesium chloride, which is known to have a somewhat greater affect on ionic strength. In many instances, the cross-linked carboxy-containing polymer will be a poly(acrylic acid) type polymer, e.g., the polymers referred to in the art as polycarbophil or carbomer. The preferred weight ratio of carboxy polymer to salt is about 4:1 to 20:1, or from about 6:1 to about 12:1.

The lightly cross-linked carboxy-containing polymers for use in the present invention are lightly cross-linked polymers of acrylic acid or the like and are, in general, well-known in the art. See, for example, Robinson U.S. Pat. No. 4,615,697, and International Publication No. WO 89/06964. These polymers are also described by Davis et al in U.S. Pat. No. 5,192,535.

In a preferred embodiments of the formulation vehicle, suitable polymers are ones prepared from at least about 90% and preferably from about 95% by weight, based on the total weight of monomers present, of one or more carboxyl-containing monoethylenically unsaturated monomers. Acrylic acid is the preferred carboxyl-containing monoethylenically unsaturated monomer, but other unsaturated, polymerizable carboxyl-containing monomers, such as methacrylic acid, ethacrylic acid, β-methylacrylic acid (crotonic acid), cis-α-methylcrotonic acid (angelic acid), trans-α-methylcrotonic acid (tiglic acid), α-butylcrotonic acid, α-phenylacrylic acid, α-benzylacrylic acid, α-cyclohexylacrylic acid, β-phenylacrylic acid (cinnamic acid), coumaric acid (o-hydroxycinnamic acid), umbellic acid (p-hydroxycoumaric acid), and the like can be used in addition to or instead of acrylic acid.

The lightly cross-linked carboxy-containing polymers are prepared by using a small percentage, i.e., less than about 5%, such as from about 0.01% or from about 0.5% to about 5%, and preferably from about 0.2% to about 3%, based on the total weight of monomers present, of a polyfunctional cross-linking agent. Included among such cross-linking agents are non-polyalkenyl polyether difunctional cross-linking monomers such as divinyl glycol; 3,4-dihydroxy-hexa-1,5-diene; 2,5-dimethyl-1,5-hexadiene; divinylbenzene; N,N-diallylacrylamide; N,N-diallylmethacrylamide and the like.

The lightly cross-linked polymers can of course be made from a carboxyl-containing monomer or monomers as the sole monoethylenically unsaturated monomer present, together with a cross-linking agent or agents. They can also be polymers in which up to about 40%, and preferably from about 0% to about 20% by weight, of the carboxyl-containing monoethylenically unsaturated monomer or monomers has been replaced by one or more non-carboxyl-containing monoethylenically unsaturated monomers containing only physiologically (and, where appropriate, ophthalmologically) innocuous substituents, including acrylic and methacrylic acid esters such as methyl methacrylate, ethyl acrylate, butyl acrylate, 2-ethylhexylacrylate, vinyl acetate, 2-hydroxyethylmethacrylate, 3-hydroxypropylacrylate, and the like. Particularly preferred polymers are lightly cross-linked acrylic acid polymers wherein the cross-linking monomer is 3,4-dihydroxyhexa-1,5-diene or 2,5-dimethylhexa-1,5-diene.

An especially preferred lightly cross-linked carboxy-containing polymer for use herein is polycarbophil, particularly NOVEON AA1, a carboxyl-containing polymer prepared by suspension polymerizing acrylic acid and divinyl glycol. NOVEON AA1 (also called Carbopol 976) is commercially available from The B.F. Goodrich Company. A different preferred lightly cross-linked carboxy-containing polymer for use herein is Carbopol 974P which is prepared using a different polyfunctional cross-linking agent of the polyalkenyl polyether type. Still another class of lightly cross-linked carboxy-containing polymer are known in the art as carbomer, e.g., carbomer 940.

The lightly cross-linked polymers can be commercially available, or are generally preferably prepared by suspension or emulsion polymerizing the monomers, using conventional free radical polymerization catalysts. In general, such polymers will range in molecular weight estimated to be from about 250,000 to about 4,000,000, and preferably from about 500,000 to about 2,000,000.

As stated, the suspensions of the invention will include an ophthalmic pharmaceutical active that has a solubility in water at 25° C. and pH of 7 of less than 10% of the formulated concentration. A relatively short list of known ophthalmic actives that can be suspended in the vehicle formulation described herein includes, but not limited to, dexamethasone at concentrations of from 0.1% to 0.2% by weight, fluoromethalone at concentrations of from 0.05% to 0.25% by weight, prednisolone at concentrations of from 0.1% to 1% by weight, loteprednol etabonate at concentrations of from 0.1% to 0.5% by weight, besifloxacin at concentrations of from 0.1% to 0.6% by weight, and cyclosporine at concentrations of from 0.03% to 0.07% by weight.

One embodiment of the invention, would include a drug product, which takes advantage of the tear thin character of the vehicle formulation described herein, will include an ophthalmic active that meets the water solubility limit stated above and is non-steroidal, that is, the active is not a steroid or soft-steroid as that term is well defined in the art of ophthalmic formulations and drug products. Exemplary pharmaceutical actives include any well known, or to be developed, active for the treatment of dry eye, allergy, glaucoma, inflammation and infection.

In another embodiment of the invention, a drug product, which takes advantage of the tear thin character of the vehicle formulation described herein, will include an ophthalmic pharmaceutical active that meets the water solubility limit stated above, and is a steroid or soft steroid as that term is well defined in the art of ophthalmic formulations and drug products. Of such class of drug products would include a suspension of Loteprednol Etabonate (at times referred to as LE), (11β,17α),-7-((Ethoxycarbonyl)oxy)-11-hydroxy-3-oxoandrosta-1,4-diene-17-carboxylic acid chloromethyl ester is a known compound and can be synthesized by methods disclosed in U.S. Pat. No. 4,996,335, the entire contents of which are hereby incorporated by reference in the present specification. A preferred concentration of LE in the described suspensions is from 0.1 wt. % to 0.25 wt. %, more preferably from 0.14 wt. % to 2.0 wt. %.

LE (0.5%) is indicated as an ophthalmic anti-inflammatory agent. LE has a solubility in water of about 0.008 mg/mL. The recommended administration dosage is one drop to each eye (0.25 mg/eye), 4× (four times) daily, for a total dosage of 1.0 mg/eye/day. LE (0.2%) is indicated for temporary relief of signs and symptoms of Seasonal Allergic Conjunctivitis (SAC). The recommended administration dosage is one drop to each eye (0.1 mg/eye), 4× (four times) daily, for a total dosage of 0.4 mg/eye/day, or one drop to each eye (0.1 mg/eye), 2× (two times) daily, for a total dosage of 0.2 mg/eye/day.

An improved ophthalmic formulation over the current Alrex® formulation is described. The ophthalmic formulation contains 20% less active, 0.16 wt. % loteprednol etabonate vs. the 0.2 wt. % loteprednol etabonate in the Alrex® product. More importantly, a small clinical study indicates that the ophthalmic formulation (taken once daily) is more effective in reducing ocular itching for the treatment of seasonal allergic conjunctivitis than Alrex® (taken 4× per day). In other words, a once daily, drop administration of the ophthalmic formulation (0.16 wt. %) is more effective than 4×0.2 wt. % for a total administration of 0.8 wt. % of Alrex®. This is a very significant achievement as a patient has no need to administer additional drops to the eye other than once in the morning or evening, thereby significantly improving upon patient compliance and convenience. In addition, unlike the aqueous suspension Alrex® the ophthalmic formulation is non-settling, and therefore, does not require vigorous repeated shaking prior to installation, which again leads to greater patient compliance and greater convenience for the patient. Accordingly, the invention is directed to a method of treating allergic conjunctivitis comprising instructing a person suffering from ocular itching resulting from allergic conjunctivitis to administer once daily in the form of one or more eye drops an aqueous ophthalmic formulation just described. The once daily administration of this LE formulation has greater clinical efficacy than if the prior art LE suspension is administered twice or four-times daily.

In general, the present invention provides an ophthalmic formulation that is topically administrable into an eye of a subject as a drop. In one embodiment, the viscosity of the vehicle formulation does not increase upon contact with the tear fluid in the eye. The vehicle formulation is sufficiently viscous (>1000 cps at 7.5 s⁻¹ shear) to ensure the particles of loteprednol etabonate that are suspended in the vehicle and do not settle over time. The stabilized gel formulation does not require shaking of the dosage package to re-suspend the drug particles prior to drop administration. In contrast, the drug particles in the low viscosity Alrex® formulation settle over time, and therefore, the dose package does require shaking prior to drop administration to ensure a properly uniform unit dosage.

In addition, other steroidal compounds for treating ocular inflammations can be based on predictably metabolized drugs. Predictably metabolized drugs, as is known in the art, are designed to provide maximal therapeutic effect and minimal side effects. By one approach, synthesis of a “predictably metabolized drug” can be achieved by structurally modifying a known inactive metabolite of a known active drug to produce an active metabolite that undergoes a predictable one-step transformation in-vivo back to the parent, inactive metabolite (see; e.g., U.S. Pat. Nos. 6,610,675, 4,996,335 and 4,710,495 for predictably metabolized steroids). “Predictably metabolized drugs” therefore are biologically active chemical components characterized by predictable in-vivo metabolism to non-toxic derivatives after they provide their therapeutic effect. Formulations of steroids suitable for ophthalmic use are known. For example, U.S. Pat. Nos. 4,710,495, 4,996,335, 5,540,930, 5,747,061, 5,916,550, 6,368,616 and 6,610,675, the contents of each of which is incorporated by reference herein, describe predictably metabolized steroids and/or formulations containing predictably metabolized steroids.

The vehicle formulations described herein can also include various other ingredients, including but not limited to surfactants, tonicity agents, buffers, preservatives, co-solvents and viscosity-building agents.

Surfactants that can be used are surface-active agents that are acceptable for ophthalmic or otolaryngological uses. Useful surface active agents include but are not limited to polysorbate 80, tyloxapol, Tween® 80 (ICI America Inc., Wilmington, Del.), Pluronic® F-68 (from BASF, Ludwigshafen, Germany) and the poloxamer surfactants can also be used. These surfactants are nonionic alkaline oxide condensates of an organic compound which contains hydroxyl groups. The concentration in which the surface active agent may be used is only limited by neutralization of the bactericidal effects on the accompanying preservatives (if present), or by concentrations which may cause irritation.

Various tonicity agents may be employed to adjust the tonicity of the formulation. For example, sodium chloride, potassium chloride, magnesium chloride, calcium chloride, nonionic diols, preferably glycerol, dextrose and/or mannitol may be added to the formulation to approximate physiological tonicity. Such an amount of tonicity agent will vary, depending on the particular agent to be added. In general, however, the formulations will have a tonicity agent in an amount sufficient to cause the final formulation to have an ophthalmically acceptable osmolality (generally about 150-450 mOsm/kg). A nonionic tonicity agent is preferred. However, if an ionic compound is used to assist in adjusting the tonicity, such compound is used in an amount such that the total concentration of cations in a composition of the present invention is within the range herein disclosed.

An appropriate buffer system (e.g., sodium phosphate, sodium acetate, sodium citrate, sodium borate or boric acid) may be added to the formulations to prevent pH drift under storage conditions. The particular concentration will vary, depending on the agent employed.

Topical ophthalmic products are typically packaged in multidose form. Preservatives are thus required to prevent microbial contamination during use. Suitable preservatives include: biguanides, hydrogen peroxide, hydrogen peroxide producers, benzalkonium chloride, chlorobutanol, benzododecinium bromide, methyl paraben, propyl paraben, phenylethyl alcohol edetate disodium, sorbic acid, polyquaternium-1, or other agents known to those skilled in the art. Such preservatives are typically employed at a level of from 0.001 to 1% (w/w). Unit dose forms will be sterile and generally will not contain preservatives.

Co-solvents and viscosity-building agents may be added to the formulations to improve the characteristics of the formulations. Such materials can include nonionic water-soluble polymer. Other compounds designed to lubricate, “wet,’ approximate the consistency of endogenous tears, aid in natural tear build-up, or otherwise provide temporary relief of dry eye symptoms and conditions upon ocular administration the eye are known in the art. Such compounds may enhance the viscosity of the formulation, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P.

Other compounds may also be added to the ophthalmic formulations of the present invention to increase the viscosity of the carrier. Examples of viscosity-enhancing agents include, but are not limited to: polysaccharides, such as hyaluronic acid and its salts, chondroitin sulfate and its salts, dextrans, various polymers of the cellulose family; vinyl polymers; and acrylic acid polymers.

Formulations formulated for the treatment of dry eye-type diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous formulations which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; (ii) are safe; and (iii) provide the appropriate delivery vehicle for the topical administration of an effective amount of an API for the treatment or relief of such condition. An example of such an API may be (11β,17α),-17-((ethoxycarbonyl)oxy)-11-hydroxy-3-oxoandrosta-1,4-diene-17-carboxylic acid chloromethyl ester. Examples of artificial tears formulations useful as artificial tears carriers include, but are not limited to, commercial products, such as Moisture Eyes™ Lubricant Eye Drops/Artificial Tears, Moisture Eyes™, Liquid Gel lubricant eye drops, Moisture Eyes™, Preservative Free Lubricant Eye Drops/Artificial Tears and Moisture Eyes™, Liquid Gel Preservative Free Lubricant Eye Drops/Artificial Tears (Bausch & Lomb Incorporated, Rochester, N.Y.). Examples of phospholipid carrier formulations include those disclosed in U.S. Pat. No. 4,804,539 (Guo et al.), U.S. Pat. No. 4,883,658 (Holly), U.S. Pat. No. 4,914,088 (Glonek), U.S. Pat. No. 5,075,104 (Gressel et al.), U.S. Pat. No. 5,278,151 (Korb et al.), U.S. Pat. No. 5,294,607 (Glonek et al.), U.S. Pat. No. 5,371,108 (Korb et al.), U.S. Pat. No. 5,578,586 (Glonek et al.), the contents of each of which are incorporated by reference herein.

The preferred formulations of the present invention are intended for administration to a human patient suffering from ophthalmic diseases such as dry eye or symptoms of dry eye. Preferably, such formulations will be administered topically. In general, the doses used for the above described purposes will vary, but will be in an effective amount to eliminate or improve dry eye conditions. Generally, 1-2 drops of such formulations will be administered from once to many times per day. The formulation is intended to be provided as a package for the treatment of dry eye, the package would include a pharmaceutical formulation prepared with a formulation vehicle described herein. In certain embodiments wherein the formulation is preservative free, the package would contain a pharmaceutically acceptable container suitable for single use by a user of the packaged formulation. Preferably the outer packing would contain a multiplicity of single use containers, for example, enough single use containers to provide for a one-month supply of the formulation. To minimize or prevent loss of water from the unit dosage forms the unit dosage forms can be foil wrapped into weekly package units.

The invention will now be further described by way of several examples that are intended to describe but not limit the scope of the invention as defined by the claims herein.

Representative eye drop formulations are provided by the Examples and Comparative Examples below.

### Example 1

A sterile, aqueous polyacrylic acid polymer solution is mixed with a sterile-filtrated solution of preserving agent, isotonicity agent, and chelating agent. After careful and thorough mixing of the starting materials, the addition of sterile-filtrated caustic soda solution initiates gel formation, and the gel is further subjected to agitation until it is homogenous. Meanwhile loteprednol etabonate or its pharmaceutically acceptable ester is sterilized. This can be accomplished by dissolving the active substance in a suitable amount of solvent, for example ethyl acetate, subjecting the solution to sterile filtration, and precipitating the active substance, for example, through the addition of sterile water with an anti-microbial agent under aseptic conditions. The microbially sterile loteprednol etabonate or its pharmaceutically acceptable ester is then triturated or ground to a powder with about three to ten times that amount of the polyacrylic acid gel to form a concentrate. The remaining amount of gel is then slowly incorporated into the concentrate with thorough mixing. The resulting suspension is then conventionally decanted or drawn off under sterile conditions into sterile containers. In an alternative variation of this method, the microbially sterile loteprednol etabonate or its pharmaceutically acceptable ester can be, to a large extent, suspended in a part of the aqueous solution of the tonicity agent. The polyacrylate gel can be made in a conventional manner with the remaining amount of isotonic agent and separately the isotonic suspension of the loteprednol etabonate can be homogenously mixed with the polyacrylate under sterile conditions.

The gel suspension is well acceptable to the patient because upon instillation it does not have the undesired characteristics of known ointments and is not oily. Also, stability studies have shown so that the gel has a relatively long shelf life without any change in its physical properties. In particular, there is no settlement of loteprednol etabonate from the gel upon storage (25-40° C.) for at least 16 months. In addition, no crystal growth of the active ingredient is observed. This sterile gel suspension represents a significantly improved drug dosage form for ophthalmic applications.

TABLE 2
LE Suspension Formulations

| | | | Amount Range | |
|---|---|---|---|---|
| | | | (per 100 g of total | |
| Ingredient | Ex. 1 | | composition) | |
| Cross-linked carboxy polymer) | 0.375 | g | 0.2-0.5 g; 0.3-0.4 g | |
| Purified water | 99.625 | g | q.s. to 100 g of | |
| Propylene glycol | 0.44 | g | 0.3-0.6 g; 0.4-0.5 g | |
| Glycerin | 0.88 | g | 0.6-1 | g; |
| Loteprednol etabonate | 0.5 | g | 0.3-2 g; 0.1-0.2 g | |
| Edetate disodium dihydrate | 0.055 | g | 0.03-0.07 | g |
| Tyloxapol | 0.05 | g | 0.03-1 | g |
| Boric acid | 0.5 | g | 0.3-0.6 | g |
| Sodium Chloride | 0.05 | g | 0.0-0.07 | g |
| Benzalkonium chloride (“BAK”) | 0.006 | g | 0.003-0.01 | g |

Mix the components of Example 1 for more than 15 minutes and adjust pH to 6.3-6.6 using 2N NaOH (for the foregoing formulation, about 1.6-1.7 g of 2N NaOH is adequate).

### Rheology Measurements

### Test Materials:

The rheological properties of lightly crosslinked carboxy polymer-based formulations were evaluated neat as well as following dilution with synthetic tear fluid. Hanks balanced salt solution (Gibco HBSS with calcium and magnesium, P/N 14025, Invitrogen Corp, Carlsbad, Calif.) was used as simulated tear fluid for the dilution of the formulation because it mimics the osmolality, pH, ionic strength, and buffer capacity of tear fluid and has representative levels of magnesium and calcium which could affect the viscosity of the crosslinked polyacrylic acid-based formulation upon dilution.

### Rheology Instrument:

A controlled stress rheometer (TA Instruments AR2000 with Firmware V7.20, New Castle, Del.) was used for the measurement of the rheological properties of the formulation. The measurement system was a stainless steel vaned-rotor (P/N 545025.001) and aluminum concentric cylinder cup (P/N 545622.001) which requires approximately 30 mL of sample for each measurement. The temperature of the sample cup is controlled by a peltier jacket and was maintained at 25° C. for all the experiments. Data was collected using Rheology Advantage software V5.7.13 (TA Instruments, New Castle, Del.). The measurement gap was set to 4 mm and the gap closing method was set to ‘exponential’. After closing the gap, the sample was equilibrated for 10 minutes prior to running each experiment. Data was collected using Rheology Advantage software V5.7.13 (TA Instruments, New Castle, Del.).

### Oscillatory Frequency Sweep

A frequency sweep experiment was performed at a constant oscillatory strain of 1%, by scanning from 50 to 0.2 rad/s (log scale, 10 points/decade). Vehicle formulations comprised of crosslinked polyacrylic acid polymers generally have values of G′, δ, and tan δ that are relatively constant over this frequency range. For the characterization of the gel properties in the formulation or the tear mixture, the values of G′, δ, and tan δ at 1 rad/s are used.

### Steady State Flow

A steady-state flow experiment was performed by scanning the shear rate from 100 s⁻¹ to 0 s⁻¹ (log scale, 10 points/decade). Steady state equilibrium was defined as 3 consecutive measurements within the tolerance window of 2%. The sample period was 5 seconds and the maximum time/point was set to 10 minutes. The motor mode was set to ‘auto’. The viscosity of the formulation or the tear mixture is significantly higher at low shear rate and lower at high shear rate because of the shear-thinning behavior exhibited by crosslinked polyacrylic acid polymers. The yield value for the formulation or the tear mixture is determined from the plot of shear stress versus shear rate. The yield value may be determined graphically, but a preferred method is to fit the shear rate versus shear stress data to the Herschel-Bulkley equation and use the best-fit yield value. Fitting of the steady-state flow data, in the 10 s⁻¹ to 0 s⁻¹ range, to the Herschel-Bulkley equation was performed using the Rheology Advantage Data Analysis Program (v.5.7.0). In the case where the best-fit yield value was <0, the yield value is reported as zero.

TABLE 3
Rheology analysis of separate laboratory
preparations of Example 1.

| Lab | Yield | G′ (Pa) | δ | tanδ | viscosity | viscosity |
|---|---|---|---|---|---|---|
| Batch No. | (Pa) | 1 rad/s | 1 rad/s | 1 rad/s | 1 s⁻¹ | 100 s⁻¹ |
| 1A | 3.44 | 17.6 | 3.85 | 0.0673 | 5782 | 185 |
| 1B | 3.54 | 19.0 | 3.63 | 0.0635 | 5889 | 187 |
| 1C | 3.99 | 19.5 | 3.71 | 0.0649 | 6479 | 200 |
| 1D | 4.10 | 20.0 | 3.54 | 0.0619 | 6716 | 208 |
| 1E | 3.95 | 20.2 | 3.06 | 0.0534 | 6558 | 202 |
| 1F | 4.16 | 20.3 | 3.60 | 0.0628 | 6731 | 208 |
| 1G | 4.15 | 20.2 | 3.50 | 0.0612 | 6709 | 209 |
| 1H | 4.52 | 22.2 | 3.58 | 0.0625 | 7308 | 224 |
| avg. | 4.0 | 19.9 | 3.56 | 0.0620 | 6552 | 203 |
| std. dev. | 0.3 | 1.3 | 0.23 | 0.004 | 491 | 12 |

### Example 2

TABLE 4

| Ingredient (mg/g) | Ex. 2 | Comp. Ex. 1 |
|---|---|---|
| Polycarbophil Noveon ® AA-1 | 3.75 | — |
| Propylene glycol | 4.4 | — |
| Glycerin | 8.8 | 25.0 |
| Loteprednol etabonate | 1.6 | 2.0 |
| EDTA | 0.55 | 0.1 |
| Benzalkonium chloride | 0.06 (30 ppm) | 0.2 (100 ppm) |
| Tyloxapol | 0.5 | 3.0 |
| Boric acid | 5.0 | — |
| Sodium chloride (NaCl) | 0.5 | — |
| Povidone K90 | — | 15.0 |

Mix the components of Example 2 for more than 15 minutes and adjust pH to 6.3-6.6 using 2N NaOH. The formulation has a calculated ionic strength of about 0.069, a tan δ of 0.10 at 10 rad/s and osmolality of about 275 mOsm/kg. The formulation also has a weight ratio of polycarbophil:NaCl of 7.5:1. Similarly, Comparative Example 1 (CE1) is prepared by mixing the listed components. CE1 is adjusted to a pH of 5.5 with hydrochloric acid. CE1 has a calculated ionic strength of about 0.001, and osmolality of about 280 mOsm/kg.

### Comparative Example 2

Comparative Example 2 (CE2) is a commercially marketed anti-infective ophthalmic composition sold as Besivance™ by Bausch & Lomb. The vehicle formulation is based on a commercial vehicle known as DuraSite®. CE2 has 8.5 mg/g of polycarbophil and 5.0 mg/g of sodium chloride. CE2 has a calculated ionic strength of about 0.236, a tan δ of 0.11 at 10 rad/s and osmolality of about 285 mOsm/kg. CE2 also has a weight ratio of polycarbophil:NaCl of 1.7:1.

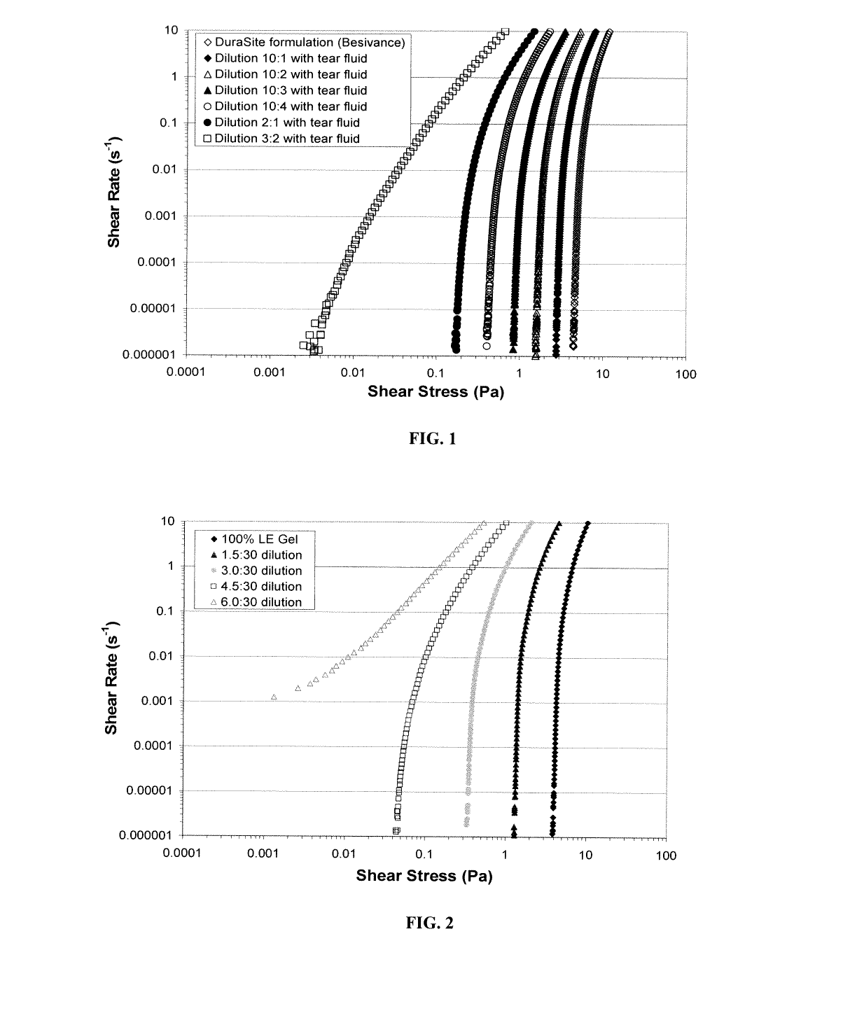

One primary difference in the rheological properties of a suspension of the invention, e.g., Example 2, and a suspension in the prior art, e.g., CE2, is demonstrated by the viscoelastic shear data in FIGS. 1 and 2. FIG. 1 shows the shear data for Comp. Ex. 2 (sample A) and various dilutions of Comp. Ex. 2 (samples B thru G) in simulated tear fluid. The tear fluid dilutions are listed for reference in the table that follows. From the data reported in FIG. 1 one first recognizes that the suspension, which is based on a commercial vehicle formulation known as DuraSite®, continues to display gel character at a dilution of 60 vol. % suspension/40 vol. % tear (dilution G). The yield value for dilution G is 0.003 Pa. One also makes note that the yield value of the undiluted suspension (sample A) is 4.5 Pa.

| CE2 dilution sample | A | B | C | D | E | F | G |
|---|---|---|---|---|---|---|---|
| vol. % suspension | 100 | 91 | 83 | 77 | 71 | 67 | 60 |
| yield value (Pa) | 4.5 | 2.8 | 1.6 | 0.87 | 0.42 | 0.18 | 0.003 |

FIG. 2 shows the shear data for Example 2 (sample A) and various dilutions of Example 2 (B thru E) in simulated tear fluid. The tear fluid dilutions are listed for reference in the table that follows. From the data reported in FIG. 2 one first recognizes that the suspension continues to display gel character at a dilution of about 87 vol. % suspension/13 vol. % tear (dilution D). The yield value for dilution D is 0.05. However, upon further dilution with very small amounts of additional tear fluid one observes a transition from a gel to liquid character of the suspension. In fact, at a dilution of 83 vol. % suspension/17 vol. % tear the suspension has lost its gel character, and its yield value is no longer measurable using the methods described herein. One also makes note that the yield value of the undiluted Ex. 2 suspension (sample A) is 3.9 Pa, which is very similar to the non-diluted yield value of the CE2 suspension (sample A). It is also important to understand that the suspension of Example 2 is storage stable meaning that the drug product need not be shaken prior to drop instillation to ensure consistent dosing throughout product use life.

| Ex. 2 dilution sample | A | B | C | D | E |
|---|---|---|---|---|---|
| vol. % suspension | 100 | 95 | 91 | 87 | 83 |
| yield value (Pa) | 3.9 | 1.3 | 0.34 | 0.049 | n.m. |

n.m.—not measurable

The pharmacokinetic properties of suspensions based upon the formulation vehicle of Example 2 (with varying concentrations of loteprednol etabonate (LE)) were investigated in vivo following topical ocular administration to rabbits. The distribution of LE to specific tissues in the anterior section of the eye was assessed, along with the potential absorption of LE into the systemic circulation. The ocular and systemic pharmacokinetics of LE afforded by the vehicle formulation of Example 2 was compared with the pharmacokinetics of LE observed with ocular administration of Comparative Example 1 (CE1, LE suspension, 0.2%). In one study LE was prepared in the vehicle formulation of Example 2 at target LE concentrations of 0.2%, 0.6%, and 1%. Results from this study (study 2) were compared with the results from a previous study (study 1) in which rabbits received CE1. Dutch-Belted rabbits were used in both studies. Animals received a single topical instillation (50 μL) of the appropriate LE suspension into each eye. At predetermined intervals through 24 hr after dosing, 4 rabbits per treatment group were euthanized and samples of plasma, tear fluid, aqueous humor, conjunctiva, and cornea were obtained for analysis. Concentrations of LE were measured using LC/MS/MS methods. Non-compartmental methods were used for the pharmacokinetic analysis of composite mean concentration versus time data.

Inter-animal variability in LE concentrations was large for all tissues in both studies. The resulting pharmacokinetic parameter values are shown in Table 5. The vehicle formulation of Example 2, based on LE Cₘₐₓ and AUC values, is summarized as follows. A 3-fold increase in the administered dose (i.e., from 0.1 mg/eye [0.2%] to 0.3 mg/eye [0.6%]) produced a less-than proportional increase (1.3- to 2.7-fold) in the ocular exposure to LE. A further increase in the administered dose to 0.5 mg/eye (1%) provided an additional ˜1.5-fold increase in LE AUC (but not Cₘₐₓ) for cornea and conjunctiva; however, no discernible increases were observed for tear or aqueous humor compared with the 0.3 mg/eye (0.6%) dose. The Example 2 LE suspension (the exception that LE is present at 0.2%) afforded higher ocular exposure (based on Cₘₐₓ or AUC) in tear and conjunctiva compared with CE1 LE suspension (LE, 0.2%). The measured exposure in cornea and aqueous humor was similar for the two LE suspensions. See, Table 4.

Systemic exposure to LE following topical ocular administration of the Example 2 LE suspension was very low, consistent with that observed with yet another LE suspension (0.5 wt. %). Specifically, following a single topical ocular administration of Example 2 based at concentrations of 0.2-1%, plasma LE concentrations were <1 ng/mL in most (125 out of 128) animals. LE concentrations of 1.01, 1.12, and 4.07 ng/mL were observed in the 3 animals with concentrations above 1 ng/mL.

TABLE 5
Pharmacokinetic Parameter Values for Loteprednol Etabonate following
a Single Topical Ocular Administration to Pigmented Rabbits

| Formu- | | Tissue/ | Cₘₐₓ^{a} | AUC₍₀₋ₜ₎ |
|---|---|---|---|---|
| lation | Dose | Matrix | (μg/g) | (μg*h/g) |
| LE | 0.1 mg/eye | Tear | 1120 ± 337 | 594 |
| Suspen- | (0.2%) | Conjunctiva | 6.96 ± 6.00 | 29.6 |
| sion | | Cornea | 1.11 ± 0.570 | 4.20 |
| (Ex. 2 | | Aq. Humor^{b} | 0.0137 ± 0.0120 | 0.0248 |
| vehicle) | 0.2 mg/eye | Tear | 1050 ± 1060 | 802 |
| | (0.4%) | Conjunctiva | 14.6 ± 15.9 | 75.8 |
| | | Cornea | 2.09 ± 0.438 | 9.77 |
| | | Aq. Humor^{b} | 0.0157 ± 0.00395 | 0.0317 |
| | 0.3 mg/eye | Tear | 2780 ± 707 | 1590 |
| | (0.6%) | Conjunctiva | 13.8 ± 5.46 | 69.8 |
| | | Cornea | 2.50 ± 1.51 | 7.87 |
| | | Aq. Humor^{b} | 0.0173 ± 0.00340 | 0.0404 |
| | 0.5 mg/eye | Tear | 2800 ± 1500 | 1490 |
| | (1%) | Conjunctiva | 18.5 ± 15.5 | 107 |
| | | Cornea | 2.74 ± 1.53 | 12.3 |
| | | Aq. Humor^{b} | 0.0191 ± 0.00876 | 0.0438 |
| Comp. | 0.1 mg/eye | Tear | 433 ± 444 | 276 |
| Ex. 1 | (0.2%)^{c} | Conjunctiva | 2.45 ± 1.59 | 22.8 |
| | | Cornea | 1.46 ± 0.422 | 4.02 |
| | | Aq. Humor^{b} | 0.0128 ± 0.00462 | 0.0237 |

^{a}Cₘₐₓ values represent maximum mean ± SD LE concentration
^{b}Relevant units for aqueous humor are μg/mL for Cₘₐₓ and μg*h/mL for AUC(0-t)
^{c}Comp. Ex. 1 is an aqueous suspension of LE with a viscosity of 3-10 cp.

In summary, the available ocular pharmacokinetic data for LE formulated in Example 2 (LE, 0.16 wt. %) provides similar or somewhat higher ocular exposure to LE compared with Comp. Ex. 1 (LE, 0.2 wt. %) in rabbits.

A small clinical study (approximately 100 subjects) was performed to evaluate LE ophthalmic suspension (Example 2) at different administration times/dosage (QD, BID and QID) versus Comp. Example 1 administered 4× daily (QID). Subjects were randomized according to a computer-generated randomization list to the following treatment groups at Visit 2. Subjects were asked to rate the comfort of the study medication drop in each eye at the time of instillation and at 1 and 2 minutes after instillation using a unitary 0-10 scale where 0 is very comfortable and 10 is very uncomfortable. Each subject received a masked envelope with instructions to follow one of the following dosing regimens. The subjects were to follow the dosing regimen for two weeks.
1. QD: Apply 1 drop in each eye once a day in the morning (t=0).
2. BID: Apply 1 drop in each eye twice a day, once in the morning (t=0) and once approximately 8 hours later (t=8).
3. QID: Apply 1 drop in each eye four times a day, one in the morning (t=0), a second drop four hours later (t=4), a third drop approximately 8 hours after the first (t=8), and a fourth drop approximately 12 hours after the first (t=12).
   The number of subjects in each test group follows.

### Example 2: QD, N=21; BID, N=18; QID, N=19.

Comp. Ex. 1: QID; N=19.
Vehicle (Ex.2, no LE); randomized at QD, BID and QID, N=19.

The primary clinical efficacy evaluation of this study was the determination of superiority of LE ophthalmic suspension (Example 2) over vehicle-treated eyes using modified CAC models (see below). A mean difference of 1.0 unit for ocular itching and conjunctival hyperemia is to be considered clinically significant at a time point. Secondary efficacy endpoints for ocular itching and conjunctival redness would be evaluated by the investigator at Visit 3 (following the 14 day test period). Clinical assessment of ocular itching is well accepted by the industry, the US FDA and the medical community in studying seasonal and perennial allergies. Ocular itching generally manifests within 3 to 5 minutes of allergen challenge in the CAC model. See, Abelson, M. B. et al., in The Ocular Surface, July 2003, 1(3), 38-60. Ocular itching was evaluated by the subject at 3, 5 and 7 minutes post challenge using a 0-4 numerical scale. Zero being none, 2.0 being a mild continuous itch without a desire to rub, and 4.0 as an incapacitating itch with an irresistible urge to rub.

Likewise, clinical assessment of conjunctival hyperemia is well accepted in the medical community. Conjunctival hyperemia generally manifests within 10 minutes of allergen challenge in the CAC model. See, Spangler, D. L. et al., Clin. Ther. August 2003, 25(8), 2245-67. Conjunctival hyperemia was evaluated by the investigator at 7, 15 and 20 minutes post challenge using a 0-4 numerical scale. Zero being none, 2.0 being moderate with apparent dilation of blood vessels, and 4.0 being extremely severe with large and numerous dilated blood vessels characterized by severe deep red color.

Secondary analysis of ocular itching scores was conducted on the ITT population with LOCF comparing LE ophthalmic suspension (with Ex.2 vehicle), 0.16%-treated subjects (QD, BID, or QID) with LE ophthalmic suspension (CE1, 0.2%-treated subjects at the Visit 4 8-hour re-challenge. Treatment effects were compared at each time point using an ANOVA model with Dunnett's adjustment, as well as a Wilcoxon rank-sum test for supportive analyses.

### Example 3

### Small Clinical Study for Ocular Itching

A LE suspension (LE 0.16 wt. % with Ex.2 vehicle), hereafter Example 3, treated subjects demonstrated lower overall ocular itching scores than did CE1 (LE 0.2 wt. %) ophthalmic suspension treated subjects at all post-CAC time points against vehicle (except 5 minutes post CAC in the BID group, at which time scores with Ex. 3 were only 0.01 unit higher) at the Visit 4 8-hour re-challenge. The mean differences in ocular itching scores between Ex. 3 and CE1 at 3, 5, and 7 minutes were as follows: QD group were −0.46, −0.49, and −0.50, respectively; in the BID group, −0.10, 0.01, and −0.01, respectively; and in the QID group, −0.11, −0.16, and −0.15, respectively.

There were no statistically significant differences between Example 3 group (QD, BID, or QID) and CE1 group at any post CAC time point, using either the ANOVA model or the Wilcoxon rank-sum test, at the Visit 4 8-hour re-challenge for the endpoint of ocular itching. Supportive analyses using an ANCOVA model confirmed that there were no statistically significant differences between Example 3 group (QD, BID, or QID) and the CE1 group (QID) at the Visit 4 8-hour re-challenge for the endpoint of ocular itching.

The descriptive statistics for the primary analysis of ocular itching scores for the Example 3 treated subjects vs. vehicle treated subjects at the Visit 4 8-hour re-challenge are provided in Table 6 and the data is summarized in Table 7.

TABLE 6
Primary Ocular Itching Scores.

| | Ex. 3 QD (N = 21) | | | Ex. 3 BID (N = 20) | | |
|---|---|---|---|---|---|---|
| time | 3 Min | 5 Min | 7 Min | 3 Min | 5 Min | 7 Min |
| Mean | 1.48 | 1.63 | 1.54 | 1.84 | 2.13 | 2.03 |
| (SD)* | (1.040) | (1.036) | (1.035) | (0.978) | (0.988) | (1.097) |
| Median | 1.25 | 1.75 | 1.75 | 1.63 | 2.25 | 2.25 |
| (range)* | (0.0-4.0) | (0.0-3.0) | (0.0-3.0) | (0.3-3.8 | (0.0-4.0) | (0.0-4.0) |
| Mean | −1.21 | −1.15 | −1.17 | −0.85 | −0.65 | −0.69 |
| difference** | | | | | | |

| | | |
|---|---|---|
| difference | −1.22 | −0.78 |
| (P value) | (<0.0001) | (0.0080) |

| | Ex. 3 QID (N = 18) | | | CE1 QID (N = 19) | | |
|---|---|---|---|---|---|---|
| time | 3 Min | 5 Min | 7 Min | 3 Min | 5 Min | 7 Min |
| Mean | 1.82 | 1.96 | 1.89 | 1.93 | 2.12 | 2.04 |
| (SD)* | (0.954) | (0.960) | (0.993) | (0.794) | (0.918) | (0.951) |
| Median | 1.75 | 2.13 | 2.00 | 2.00 | 2.25 | 2.25 |
| (range)* | (0.0-3.0) | (0.0-3.0) | (0.0-3.5) | (0.5-3.0) | (0.3-4.0) | (0.3-3.5) |
| Mean | −0.86 | −0.82 | −0.82 | — | — | — |
| difference** | | | | | | |

| | | |
|---|---|---|
| difference | −0.83 | — |
| (P value) | (0.0062) | |

*Based on 0-4 scale, where 0 = no itching and 4 = incapacitating itch with an irresistible urge to run
**The mean difference is calculated as the mean of LE gel, 0.16%—the mean of vehicle.

TABLE 7
Ocular Itching: Summary of Clinical Results (Visit 4).

| | Ex. 3 QD | | | Ex. 3 BID | | | Ex. 3 QID | | |
|---|---|---|---|---|---|---|---|---|---|
| Time (min.) | 3 | 5 | 7 | 3 | 5 | 7 | 3 | 5 | 7 |
| Mean | −1.21 | −1.15 | −1.17 | −0.85 | −0.65 | −0.69 | −0.86 | −0.82 | −0.82 |
| difference | | | | | | | | | |
| Statistical | yes | yes | yes | yes | no | no | yes | yes | yes |
| significance | | | | | | | | | |
| Clinical | yes | yes | yes | no | no | no | no | no | no |
| significance | | | | | | | | | |

| | | | |
|---|---|---|---|
| Overall | Yes | No | No |
| clinical | | | |
| success | | | |

### Example 4

A vehicle formulation of the invention that includes mapracorat is described in Table 8.

TABLE 8

| | Comp Ex 3 | Example 4 |
|---|---|---|
| Ingredient | (mg/g) | (mg/g) |
| mapracorat | 10 | 10 |
| mannitol | 39 | 46 |
| sodium borate | 7.0 | — |
| carbomer 980 | 6.0 | 3.0 |
| poloxamer 407 | 2.0 | 2.0 |
| potassium sorbate | 2.5 | — |
| disodium edetate | 0.5 | — |
| citric acid monohydrate | — | 0.8 |
| sodium hydroxide (2N) | q.s. to pH 6 | q.s. to pH 6 |
| water for injection | q.s. to 1 g | q.s. to 1 g |
| ionic strength | 0.103 | 0.058 |

TABLE 9
Rheology analysis of formulations

| | Yield | G′ (Pa) | δ | tanδ | viscosity | viscosity |
|---|---|---|---|---|---|---|
| Example | (Pa) | 1 rad/s | 1 rad/s | 1 rad/s | 1 s⁻¹ | 100 s⁻¹ |
| CE2 | 4.53 | 20.9 | 5.24 | 0.0916 | 7580 | nd |
| CE3 | 5.47 | 20.2 | 2.74 | 0.0478 | 8051 | 205 |
| 4 | 4.90 | 18.85 | 3.54 | 0.0618 | 7363 | 195 |

To determine the tear thinning characteristics of each example formulation listed in Table 9 and Example 1A, 30 mL of each formulation was mixed with a volume of tear fluid. Examples 1A and 4 were mixed with 10 mL of simulated tear fluid, and CE2 and CE3 were mixed with 12 mL of simulated tear fluid. As indicated in Table 10, although CE2 and CE3 were mixed with 20% more tear fluid these formulation did not thin, i.e., one does not observe the transition from gel to liquid. As stated, it is believed that the thinning characteristics of the described vehicle formulations is driven primarily by the differences in the ionic strength between the vehicle formulation, which has a relatively low ionic strength, and the relatively high ionic strength of tear fluid, represented by the simulated tear fluid. As the vehicle formulation is administered in the form of an eye drop onto an eye, the percentage increase in the ionic strength of the vehicle-tear mixture causes the vehicle formulation to thin. Table 10 lists the concentration of the lightly cross-linked carboxy polymer, the calculated ionic strength of the example (vehicle) formulation, and the percent ratio in ionic strengths of the vehicle formulation over the simulated tear fluid given by the equation below. The ionic strength of the simulated tear fluid is 0.154. Because the concentration of polymer also play s a role in the thinning characteristics of the vehicle formulations one can multiply the polymer concentration by the % ionic strength to give a tear thin value that can be used to predict with some confidence whether one will observe the desired tear thinning of a vehicle formulation. This value is also listed in Table 10.

% ionic strength=[formulation i.s./tear i.s.]×100

tear thin value=% ionic strength×[polymer, wt. %]

TABLE 10
Rheology analysis of formulations with tear dilution

| | Yield | G′ (Pa) | δ | tanδ | viscosity | viscosity |
|---|---|---|---|---|---|---|
| Example | (Pa) | 1 rad/s | 1 rad/s | 1 rad/s | 1 s⁻¹ | 100 s⁻¹ |
| 1A | 0 ^{a} | 0.017 | 29.9 | 0.576 | 21.5 | 8.46 |
| CE2 | 0.423 | 2.70 | 6.90 | 0.121 | 1123 | nd |
| CE3 | 0.268 | 1.60 | 4.98 | 0.0871 | 696 | 35.2 |
| 4 | 0 ^{a} | 0.002 | 59.9 | 1.726 | 10.9 | 5.27 |

^{a} could not be measured

TABLE 11
Percent difference in ionic strength

| | [polymer] | | % ratio | tear thin |
|---|---|---|---|---|
| Example | wt. % | i.s. | i.s. | value |
| 1A | 0.375 | 0.069 | 45 | 16.9 |
| CE2 | 0.85 | 0.236 | 153 | 130 |
| CE3 | 0.6 | 0.103 | 67 | 40.2 |
| 4 | 0.30 | 0.058 | 38 | 11.3 |

Some of the more preferred vehicle formulations of the invention will have a % ionic strength of 60% or less, e.g., from 20% to 60%, or a tear thin value of 30 or less, e.g., from 5 to 30, or from 10 to 25.

### Stability of Suspension

The vehicle formulations exhibit excellent stability upon long-term storage. The pharmaceutical active remains suspended in the vehicle upon storage at 25° C. and 40° C. for up to 16 months and longer. Formulations were prepared according to Example 1 that had target concentrations of loteprednol etabonate of 2-6 mg/g. Samples of the formulation were stored in polyethylene dropper bottles at 25° C. and 40° C. The concentrations of loteprednol etabonate of two samples and of aliquots of the same drawn from the top and bottom of a bottle were measured after 16 months of undisturbed storage. The concentrations were determined in duplicate by liquid chromatography, as known in the art, and are presented in Table 12.

TABLE 12
Settling behavior of loteprednol etabonate ophthalmic gel

| Lot No./ | Storage | Settling | Sample | % Label |
|---|---|---|---|---|
| Concentration | Temperature | Conditions | Location | Claim (LE) |
| 151-1 | 25° C. | At rest for | Top | 104.0% |
| (2 mg/mL) | | 16 months | Bottom | 103.2% |
| 151-2 | 25° C. | At rest for | Top | 103.8% |
| (4 mg/mL) | | 16 months | Bottom | 105.4% |
| | 40° C. | At rest for | Top | 100.8% |
| | | 16 months | Bottom | 100.4% |
| 151-3 | 25° C. | At rest for | Top | 102.7% |
| (6 mg/mL) | | 16 months | Bottom | 104.4% |
| | 40° C. | At rest for | Top | 107.6% |
| | | 16 months | Bottom | 106.7% |

The foregoing data show that compositions of the present invention exhibit both good chemical and physical stability upon long-term storage even at an aggressive temperature condition. The active ingredient loteprednol etabonate continued to be suspended uniformly throughout the container. In other words, there is no settling of loteprednol etabonate from the vehicle and there is no indication of any loss of potency for the active ingredient.

### Example 5

A single unit dosage package that includes cyclosporine 0.05 wt. % in a formulation vehicle that includes 0.35 wt. % carbomer 980, polysorbate 80, mannitol and glycerin. The unit dosage form is used to administer the cyclosporine via eye drop to each eye once or twice a day. The cyclosporine is effectively suspended in the unit dosage form for up to six months.

The invention is also directed to a suspension comprising an ophthalmic active that has a solubility in water at 25° C. and a pH of 7 of less than 0.1 times the concentration of the active in mg/mL in the suspension, and the ophthalmic active is suspended in a formulation vehicle. The vehicle comprises a lightly crosslinked carboxy-containing polymer and a concentration of ionic salt components to provide the suspension with a calculated ionic strength of from 0.03 to 0.08. The suspension has the following rheological properties, G′>G″ and a suspension yield value of from 2 Pa to 8 Pa. Also upon addition of 30 mL of the suspension to a volume of 10 mL of simulated tear fluid one forms a tear mixture of the suspension, which is used to simulate the ocular environment following the instillation of one or more drops to a human eye. The tear mixture has a tear mixture yield value from 0 Pa to 0.1 Pa and a tear thin value of from 5 to 30.

In one instance, the ophthalmic active is loteprednol etabonate, which is present at a concentration of from 0.1 wt. % to 0.25 wt. %. In another instance, the ophthalmic active is non-steroidal. In either instance, the suspension will include a carboxy-containing polymer selected from the group consisting of polycarbophil and carbomer. An exemplary suspension will include a formulation vehicle that comprises 0.2-0.5% of the carboxy-containing polymer, 0.3-0.6% propylene glycol, 0.6-1% glycerin, and water, wherein all percentages are in percent by weight of the suspension. In many instances, the suspension will have a tan δ measured at 1 rad/s of from 0.035 to 0.105.

This invention has been described by reference to certain preferred embodiments; however, it should be understood that it may be embodied in other specific forms or variations thereof without departing from its special or essential characteristics. The embodiments described above are, therefore, considered to be illustrative in all respects and not restrictive, the scope of the invention being indicated by the appended claims rather than by the foregoing description.

1. A suspension comprising an ophthalmic active that has a solubility in water at 25° C. and pH of 7 of less than 0.1 times the concentration of the active in mg/mL in the suspension, the ophthalmic active suspended in a formulation vehicle, the vehicle comprising a lightly cross-linked carboxy-containing polymer and a concentration of ionic salt components to provide the suspension with a calculated ionic strength of less than 0.1,
wherein the suspension has the following rheological properties, G′>G″ and a suspension yield value of greater than 1 Pa, and upon addition of 30 mL of the suspension to a volume of 6 mL to 12 mL of simulated tear fluid to provide a tear mixture of the suspension in a simulated ocular condition, the tear mixture has G″>G′ and a tear mixture yield value of less than 0.1 Pa.

2. The suspension of claim 1 wherein the ophthalmic active is loteprednol etabonate.

3. The suspension of claim 1 wherein the ophthalmic active is non-steroidal.

4. The suspension of claim 1 wherein the carboxy-containing polymer is selected from the group consisting of polycarbophil and carbomer.

5. The suspension of claim 1 wherein the suspension has a tan δ measured at 1 rad/s of from 0.035 to 0.105.

6. The suspension of claim 1 wherein the suspension has a yield value of from 2 Pa to 10 Pa.

7. The suspension of claim 2 comprising 0.2-0.5% of the carboxy-containing polymer, 0.3-0.6% propylene glycol, 0.6-1% glycerin, 0.1-0.25% loteprednol etabonate and water, wherein all percentages are in percent by weight of the total composition.

8. The suspension of claim 7 wherein the loteprednol etabonate is present at a concentration of from 0.14% or 0.2%.

9. The suspension of claim 1 wherein the tear mixture yield value is from 0 Pa to 0.05 Pa if 30 mL of the suspension is diluted with a volume of 6 mL of simulated tear fluid.

10. The suspension of claim 1 wherein the tear mixture has no measurable yield value if 30 mL of the suspension is diluted with a volume of 10 mL of simulated tear fluid.

11. The suspension of claim 1 wherein the 30 mL of the suspension is diluted with a volume of 10 mL of simulated tear fluid providing the mixture with a tear thin value of from 5 to 30.

12. The suspension of claim 11 wherein the tear thin value is from 10 to 25.

13. A suspension comprising an ophthalmic active that has a solubility in water at 25° C. and a pH of 7 of less than 0.1 times the concentration of the active in mg/mL in the suspension, and the ophthalmic active is suspended in a formulation vehicle, the vehicle comprising a lightly crosslinked carboxy-containing polymer and a concentration of ionic salt components to provide the suspension with a calculated ionic strength of from 0.03 to 0.08, wherein the suspension has the following rheological properties, G′>G″ and a suspension yield value of from 2 Pa to 8 Pa, and upon addition of 30 mL of the suspension to a volume of 10 mL of simulated tear fluid to provide a tear mixture of the suspension in a simulated ocular condition, the tear mixture has a tear mixture yield value from 0 Pa to 0.1 Pa and a tear thin value of from 5 to 30.

14. The suspension of claim 13 wherein the ophthalmic active is loteprednol etabonate, which is present at a concentration of from 0.1 wt. % to 0.25 wt. %.

15. The suspension of claim 13 wherein the ophthalmic active is non-steroidal.

16. The suspension of claim 13 wherein the carboxy-containing polymer is selected from the group consisting of polycarbophil and carbomer.

17. The suspension of claim 14 wherein the formulation vehicle comprises 0.2-0.5% of the carboxy-containing polymer, 0.3-0.6% propylene glycol, 0.6-1% glycerin, and water, wherein all percentages are in percent by weight of the suspension.

18. The suspension of claim 13 wherein the suspension has a tan δ measured at 1 rad/s of from 0.035 to 0.105.

19. A method for suspending an ophthalmic active that has a solubility in water at 25° C. and a pH of 7 of less than 0.1 times the concentration of the active in mg/mL in an aqueous-based, ophthalmic suspension, the method comprising:
combining the ophthalmic active with a formulation vehicle, the vehicle comprising a lightly crosslinked carboxy-containing polymer and a concentration of ionic salt components to provide the suspension with a calculated ionic strength of from 0.03 to 0.08, wherein the suspension has the following rheological properties, G′>G″, a suspension yield value of from 2 Pa to 8 Pa, and upon addition of 30 mL of the suspension to a volume of 10 mL of simulated tear fluid to provide a tear mixture of the suspension in a simulated ocular condition, the tear mixture has a tear mixture yield value of less than 0.1 Pa and a tear thin value of from 5 to 30.

20. The method of claim 19 wherein the tear mixture yield value is from 0 Pa to 0.05 Pa and a tear thin value of from 10 to 25.

21. The method of claim 19 wherein the suspension has a tan δ measured at 1 rad/s of from 0.035 to 0.105.

22. A unit dosage package for administration of an ophthalmic formulation in the form of an eye drop, the ophthalmic formulation comprising an ophthalmic active that has a solubility in water at 25° C. and a pH of 7 of less than 0.1 times the concentration of the active in mg/mL in the formulation, wherein the ophthalmic active is suspended in a formulation vehicle, the formulation vehicle comprises a lightly crosslinked carboxy-containing polymer and a concentration of ionic salt components to provide the suspension with a calculated ionic strength of from 0.03 to 0.08, wherein the ophthalmic formulation has the following rheological properties, G′>G″, and a suspension yield value of from 2 Pa to 8 Pa, and upon addition of 30 mL of the suspension to a volume of 10 mL of simulated tear fluid to provide a tear mixture of the suspension in a simulated ocular condition, the tear mixture has a tear mixture yield value from 0 Pa to 0.1 Pa and a tear thin value of from 5 to 30.

23. The unit dosage form of 22 wherein the ophthalmic active is loteprednol etabonate, which is present from 0.1 wt. % to 0.25 wt. %.

24. The suspension of claim 22 wherein the ophthalmic active is non-steroidal.
